(19) United States
(12) Patent Application Publication
(10) Pub. No.: US 2025/0140349 A1
(43) Pub. Date: May 1, 2025

(54) CONDITIONAL GENERATION OF PROTEIN SEQUENCES

(71) Applicant: Microsoft Technology Licensing, LLC, Redmond, WA (US)

(72) Inventors: Bruce James WITTMANN, Redmond, WA (US); Eric J. HORVITZ, Freeland, WA (US); Rohan Vishesh KOODLI, Saratoga, CA (US)

(73) Assignee: Microsoft Technology Licensing, LLC, Redmond, WA (US)

(21) Appl. No.: 18/495,662

(22) Filed: Oct. 26, 2023

(51) Int. Cl. G16B 30/20; G16B 5/20

(52) U.S. Cl. CPC G16B 30/20; G16B 5/20

(57) ABSTRACT

A computing system for conditional generation of protein sequences includes processing circuitry that implements a denoising diffusion probabilistic model. In an inference phase, the processing circuitry receives an instruction to generate a predicted protein sequence having a target functionality, the instruction including first conditional information and second conditional information. The processing circuitry concatenates a first conditional information embedding generated by a first encoder and a second conditional information embedding generated by a second encoder to produce a concatenated conditional information embedding. The processing circuitry samples noise from a distribution function and combines the concatenated conditional information embedding with the sampled noise to produce a noisy concatenated input. The processor inputs the noisy concatenated input to a denoising neural network to generate a predicted sequence embedding, inputs the predicted sequence embedding to a decoding neural network to generate the predicted protein sequence, and outputs the predicted protein sequence.

### BACKGROUND

In the field of computational protein engineering, computer-based techniques have been developed to identify a protein sequence that results in a three-dimensional protein structure having molecular properties to achieve a target molecular function, given a set of conditions that describe the function. As molecular properties can have a wide-ranging impact on the activity and function of a molecule or substrate, tools for predicting optimized protein sequences are of great interest for a wide variety of fields, including drug design and drug discovery. However, as discussed below, opportunities remain for improvements in the generation of protein sequences and protein structures, particularly with the goals of achieving specific target functional capabilities of the proteins.

### SUMMARY

To address the issues discussed herein, computing systems and methods for conditional generation of protein sequences are provided. In one aspect, the computing system includes processing circuitry that executes instructions using portions of associated memory to implement a denoising diffusion probabilistic model. In an inference phase, the processing circuitry is configured to receive an instruction to generate a predicted protein sequence having a target functionality. The instruction includes first conditional information and second conditional information associated with the target functionality of the predicted protein sequence. A first conditional information embedding generated by a first encoder and a second conditional information embedding generated by a second encoder are concatenated to produce a concatenated conditional information embedding, with the first conditional information embedding representing the first conditional information and the second conditional information embedding representing the second conditional information. Noise is sampled from a distribution function and combined with the concatenated conditional information embedding to produce a noisy concatenated input. The noisy concatenated input is input to a denoising neural network to cause the denoising neural network to generate a predicted sequence embedding. The predicted sequence embedding is input to a decoding neural network to generate the predicted protein sequence based upon the inputted predicted sequence embedding, and the predicted protein sequence is output.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows a schematic view of a computing system for conditional generation of protein sequences using a denoising diffusion probabilistic model, according to one embodiment of the present disclosure.

FIG. 2 shows a schematic view of a training phase for the computing system of FIG. 1.

FIG. 3 shows a schematic view of an inference phase for the computing system of FIG. 1.

FIG. 4 shows a schematic view of a denoising loop to guide the computing system of FIG. 1 to generate an improved predicted protein sequence.

FIG. 5 shows a flowchart of a method for conditionally generating protein sequences according to an example implementation of the present disclosure.

FIG. 6 shows a schematic view of an example computing environment according to which the embodiments of the present disclosure may be implemented.

### DETAILED DESCRIPTION

The field of computational protein engineering has spanned decades and includes computational protein design and computational protein optimization. The goal of computational protein design is, given a set of conditions describing an idealized protein function, to identify a protein sequence that achieves that function. The goal of computational protein optimization is, given a set of conditions describing an idealized protein function and a protein that already performs that function, to identify a protein sequence with improved activity, where “activity” is defined as a quantifiable measure of how well a protein performs the target function.

In some of the earliest approaches, referred to as “rational design,” researchers relied on attempting to model the relationship between protein sequences and their three-dimensional structures to both design and optimize proteins for certain functions. While these rational design approaches have been applied to engineer new proteins successfully, rational design tools have several limitations. Most notably, detailed knowledge of the underlying mechanism for determining a target function of a protein is required, which is information that is often unavailable, particularly when the function of interest is new (i.e., there is no known protein with that function) or else poorly understood (e.g., the mechanistic details of the function have not been studied). Additionally, rational design approaches must contend with the combinatorial explosion inherent to searching the space of possible protein sequences. To briefly explain, protein sequences are comprised of ordered combinations of amino acids. For an average protein sequence length of 300 amino acids where each of those 300 is selected from a set of 20 possibilities, there are thus 20³⁰⁰≈10³⁹⁰ possible protein sequences, which presents a combinatorially large space that cannot be fully searched in a realistic time and thus limits the practicality of search-based methods. To overcome the challenges of searching such a large space, rational design approaches often rely on heuristics, particularly sampling strategies, to restrict the number of sequences searched. However, even with these restrictions, developing new proteins using rational design is slow.

To further address the difficulties of searching the combinatorially large protein sequence space, more recent computational protein engineering efforts have turned to generative machine learning. Unlike rational design strategies, which work by searching over the space of possibilities, generative approaches directly sample protein sequences that are believed to be enriched in the target function, thus making their computational cost invariant to the size of the search space. The earliest protein generative modeling strategies largely relied on small-scale generative models such as variational autoencoders (VAEs) and generative adversarial networks (GANs) that were trained on a small subset of protein sequences (i.e., those derived from a single protein family). Large-scale models such as long short-term memory (LSTM) models, transformers, graph neural networks (GNNs), etc., trained on enormous datasets of protein sequences were subsequently developed. While the above-mentioned generative modeling strategies were found to generate viable protein sequences, these approaches were largely unconditional. That is to say, generative models were trained to predict protein sequences that looked like their training data, as there was no way to apply other conditions after training that would modify the types of sequences produced.

As previously stated, the goal of computational protein design is to generate protein sequences conditioned on a set of target characteristics. As such, though the first generations of protein generative models overcame the challenge of searching combinatorial space, they were not very effective in computational protein design. The most recent generation of protein generative models aims to address this shortcoming by implementing denoising diffusion probabilistic models (DDPMs) as their core.

In view of the issues discussed above, a computing system utilizing a denoising diffusion probabilistic model is provided. The computing system described herein addresses the computational protein design and optimization issues discussed above by producing protein sequences based on conditions that are important for the protein engineering process, and by providing a means to optimize existing protein sequences over large combinatorial spaces.

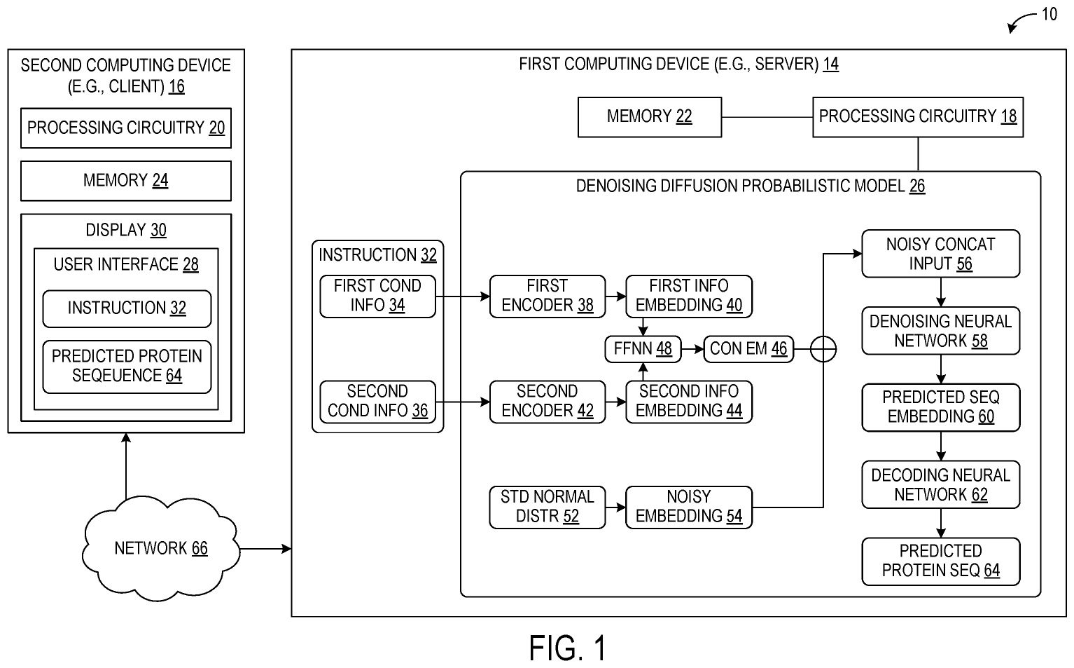

Referring initially to FIG. 1, the computing system 10 includes at least one computing device. The computing system 10 is illustrated as having a first computing device 14 including processing circuitry 18 and memory 22, and a second computing device 16 including processing circuitry 20 and memory 24. The illustrated implementation is exemplary in nature, and other configurations are possible. In the description below, the first computing device will be described as a server 14 and the second computing device will be described as a client computing device 16, and respective functions carried out at each device will be described. It will be appreciated that in other configurations, the computing system 10 may include a single computing device that carries out the salient functions of both the server 14 and client computing device 16, and that the first computing device could be a computing device other than server. In other alternative configurations, functions described as being carried out at the server 14 may alternatively be carried out at the client computing device 16 and vice versa.

Continuing with FIG. 1, the processing circuitry 18 is configured to execute instructions using portions of associated memory 22 to implement a denoising diffusion probabilistic model 26 hosted at the server 14. At a high level, the DDPM 26 is a latent diffusion model that processes conditional information to generate a protein sequence that has specific, target characteristics based on the conditional information and/or is predicted to have increased functionality with respect to another, reference protein sequence.

The client computing device 16 includes a user interface 28 that is displayed on a display 30 and configured to receive an instruction 32 input by a user. As described in detail below, this instruction 32 includes first conditional information 34 and second conditional information 36 associated with the target functionality of the predicted protein sequence, such as molecules that should bind to the generated protein, structural features that the generated protein should exhibit, information on the target expression environment for the generated protein, information on reactions that the generated protein should catalyze, and/or a free text description of the intended use and target features of the generated protein, for example. It will be appreciated that the first conditional information is different from the second conditional information.

Upon receiving the instruction 32, a first encoder 38 calculates a numerical first conditional information embedding 40 for first conditional information 34 included in the instruction 32, and a second encoder 42 calculates a numerical second conditional information embedding 44 based on the second conditional information 36. The first and second conditional information embeddings 40, 44 are concatenated into a conditional information embedding 46. Different encoders are used for different information types, or classes, of conditional information in the instruction as appropriate. For example, free text is encoded using a transformer neural network, coordinate information is encoded using a graph neural network, and so forth. The outputs of these different encoders are all combined into a single embedding using the feed forward neural network (FFNN) 48.

After computing the conditional information embedding 46, a noisy embedding 54 is sampled from a distribution function, such as a standard normal (Gaussian) distribution 52, and concatenated to the conditional embedding 46 to generate a noisy concatenated input 56. The noisy concatenated input 56 is input to a denoising neural network 58 to cause the denoising neural network 58 to generate a predicted sequence embedding 60, which is then input to a sequence decoding neural network 62 to generate the predicted protein sequence 64 based upon the inputted predicted sequence embedding 60. The predicted protein sequence 64 is output and may be viewed in the user interface 28 of the display 30 of the client computing device 16, for example. In the embodiment described herein, it will be appreciated that the server 14 is in communication with the client computing device 16 via a network 66.

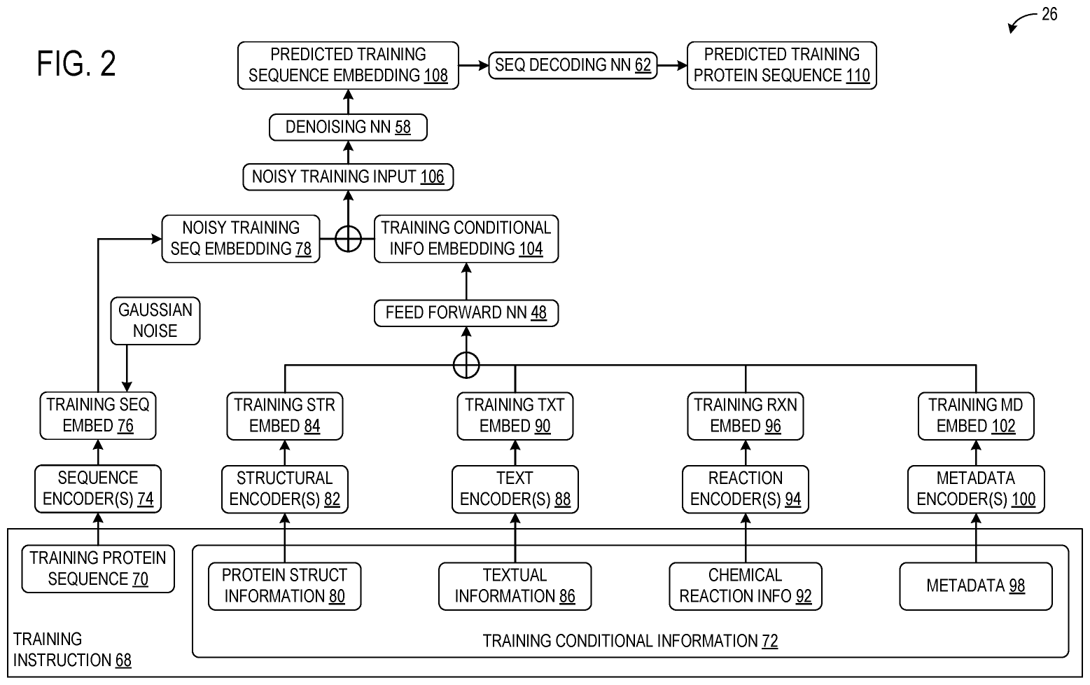

Turning to FIG. 2, an example training phase for the DDPM 26 is shown. In the training phase, a training instruction 68 to generate a predicted training protein sequence includes a training protein sequence 70 and training conditional information 72 from two or more conditional information classes. To begin, a known, i.e., training, protein sequence 70 is input into a sequence encoder 74, which may be implemented as a transformer neural network, though other encoder designs are possible, to convert the training protein sequence from raw text data to a training sequence embedding 76. The training sequence embedding 76 is configured as an L×D matrix of numbers, where L is the length of the training protein sequence and D is a chosen dimensionality. Gaussian noise is then added to the training sequence embedding 76 to produce a noisy training sequence embedding 78. The noise is added according to the schedule shown below in Equation 1:

$${x}_{t} = \sqrt{\overline{{α}_{t}}} {x}_{0} + \sqrt{1 - \overline{{α}_{t}}} ε \qquad ( 1 )$$

where x₀ is the training sequence embedding 76, xₜ is the noisy training sequence embedding 78, t is a randomly chosen integer between 1 and a predefined upper limit, T, which, in one specific example, is 2000, ε is noise drawn from a standard Gaussian distribution, and α̅ₜ̅ is a value that determines the strength of the noise added to x₀. The term α̅ₜ̅ is calculated as shown below in Equation 2:

$$\overline{{α}_{t}} = 1 - \sqrt{\frac{t}{T + s}} \qquad ( 2 )$$

where T=2000 and s is a small value added for numerical stability, although it will be appreciated other values for T could be adopted.

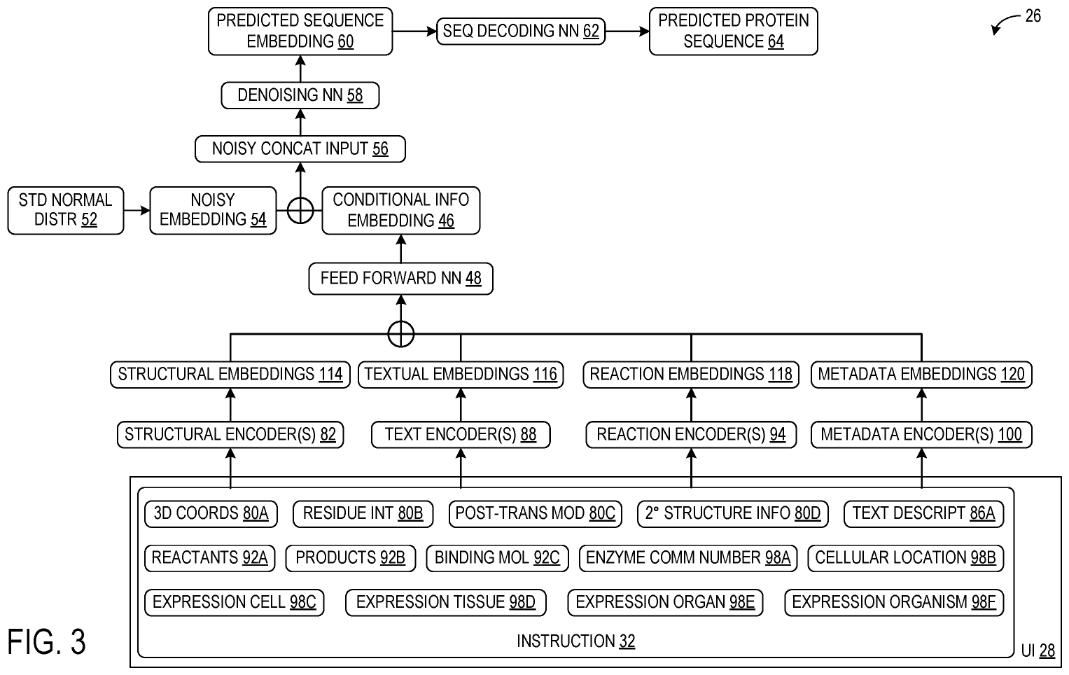

As described above with reference to FIG. 1, the predicted protein sequence is generated based on conditional information. During the training phase, the training conditional information 72 may include a diverse set of potentially relevant data, including one or more of the following: molecules that are known to bind to the training protein, structural features known to be exhibited by the training protein, information on the known expression environment of the training protein, information on known or predicted properties of the training protein, information related to the likely thermal stability of the protein or other measures of structural stability, information on reactions that the training protein is known to catalyze, and/or free text descriptions, imagery of proteins and protein interactions, and/or text linked with imagery associated with the training protein. Further description of these different categories of conditional information are provided below with reference to FIG. 3. It will be appreciated that there may be conditional information in addition to the conditional information described herein. It will be further appreciated that the conditional information may be derived from any of the categories of protein functional or structural information, textual information, chemical reaction information, and metadata associated with the training protein sequence and/or the target functionality.

Protein structural information 80 may be, for example, three-dimensional protein backbone coordinates. Each amino acid has a backbone comprising of a nitrogen (N) atom, a first carbon (Ca) atom, a second carbon (C) atom, and an oxygen (O) atom. As a protein is a chain of amino acids, a protein backbone for any given protein is formed of a repeated sequence of N—Cα—C for each amino acid in the protein, with the C atom also bonded to an O atom. The three-dimensional coordinates of the N, Cα, C, and O atoms of the training protein sequence 70 can be encoded in a variety of ways. We consider implementations that encode the three-dimensional coordinates via a message passing neural network (MPNN), which is implemented as a structural encoder 82. The resulting training structural embedding 84 has a shape L×D.

Textual information 86 may be derived from published abstracts of journal articles, captions of figures, images from figures or computational renderings, captions joined with images drawn from figures, keywords associated with the input protein sequence, and the like. Additionally or alternatively, the textual information 86 may be a natural language description of the target functionality of the protein sequence. The textual information 86 may be encoded by a generative pre-trained transformer (GPT), which is implemented as a text encoder 88 in FIG. 2. The resulting training textual embeddings 90 have a shape 1×D.

Chemical reaction information 92 may include structural characteristics of substrates and/or molecules with which atoms in the input protein structure interact. To represent these chemical structures and map chemical space, the chemical reaction information 92 is encoded using a reaction encoder 94, which may be a molecular fingerprint of reactants and products, such as the Morgan fingerprint, or a graph neural network, for example. The reaction encoder 94 converts the atom groups of all chemicals involved in an interaction with the protein into a vector having a shape 1×D, shown in FIG. 2 as a training reaction embedding 96.

Additional, less structured data (“metadata” 98) may include gene ontology terms, enzyme commission numbers, the identity of the organism expressing the protein, that organism's taxonomic identification, proteins associated with the input protein sequence that have been observed in biological systems, thermal stability of the input protein sequence, and the like. Each metadata 98 type is encoded via a separate feed forward neural network. It will be appreciated that only one of these metadata encoders 100 is depicted in FIG. 2, though in practice as many encoders are trained as there are classes of metadata. The resulting learned metadata embeddings 98 have a shape 1×D.

Conditional information is not used at every training step. Instead, each type of conditional information, when available for a given protein sequence, is included with 50% probability at each timestep. If the conditional information is not available or is unincluded, then a matrix of zeros of the appropriate shape is used in its place. The encoders 82, 88, 94, 100 may be configured to add weights to the conditional information as it is passed through a respective encoder. Additionally, one or more of the encoders may either have their weights frozen during training or unfrozen to allow for further fine-tuning of the base models. It will be further appreciated that any of the training structural embeddings 84, training textual embeddings 90, training reaction embeddings 96, and training metadata embeddings 102 may be implemented in any combination as conditional information embeddings.

Continuing with FIG. 2, the training structural embeddings 84, training textual embeddings 90, training reaction embeddings 96, and training metadata embeddings 102 are concatenated and input into the FFNN 48 to generate a training conditional information embedding 104. The training conditional information embedding 104 is combined with the noisy training sequence embedding 78 to produce a noisy training input 106.

The noisy training input 106 is input to the denoising neural network 58 to generate a predicted training sequence embedding 108. A denoising loss La is calculated as the mean square error between the predicted training sequence embedding 108 and the noisy training sequence embedding 78. The predicted training sequence embedding 108 is subsequently input to the sequence decoding neural network 62 to generate a predicted training protein sequence 110 based upon the noisy training input 106. A reconstruction loss Ly is calculated between the predicted training protein sequence 110 and the input training protein sequence 70 using cross entropy loss. The final loss for the training protein sequence is calculated as the sum of L_{d} and Lᵣ.

An example inference phase for the DDPM 26 is shown in FIG. 3. As discussed above with reference to FIG. 1, the noisy embedding 54 is sampled from a distribution function, such as the standard normal distribution 52. As with the training sequence embedding 76, this noisy embedding 54 is configured as an L×D matrix of numbers.

In the inference phase, the instruction 32 includes at least first and second conditional information 34, 36. The conditional information may be derived from protein structural information 80, textual information 86, chemical reaction information 92, and metadata 98. As shown in FIG. 3, the protein structure information may include three-dimensional coordinates 80A of the backbone atoms, desired chemical interactions 80B between different residues in the protein backbone, i.e., disulfide bonds, hydrogen bonds, etc., post-translational modifications 80C, and secondary structure information 80D, such as residues involved in alpha helices and beta sheets. The textual information 86 may include a text description 86A of the target functionality of the protein, such as a natural language description of the target functionality, abstracts gleaned from journal articles, conferences, textbooks, theses, and the like, captions of figures, and keywords, for example. Chemical reaction information 92 may include specific reactants 92A and products 92B of a chemical reaction that the protein should catalyze, and molecules 92C that are desired to bind to or interact with the protein. Metadata 98 may include an enzyme commission number 98A for the type of chemical reaction the protein should catalyze, a cellular location 98B where the protein is expressed, a cell type 98C in which the protein should be expressed, a tissue type 98D in which the protein should be expressed, an organ 98E in which the protein should be expressed, and an organism 98F in which the protein should be expressed, for example. Using any number and combination of conditional information, a user may instruct the model to predict a protein sequence for a protein that exhibits a target functionality, can be expressed in a specific location, and/or under certain conditions. As such, the DDPM 26 may incorporate conditional information for the target functionality, as well as the specified expression environment, to use in de novo protein design.

As described above with respect to FIG. 1, each class of conditional information is encoded by a respective encoder to generate at least the first and second conditional information embeddings 40, 44, which may be any of structural embeddings 114, textual embeddings 116, reaction embeddings 118, and metadata embeddings 120. It will be appreciated that the conditional information embeddings may be encoded during the training phase and/or during the inference phase, depending on whether relevant new conditional information was acquired after the training phase. The embeddings are concatenated and input into the FFNN 48 to generate the conditional information embedding 46. Additionally or alternatively, the FFNN 48 may include attention heads that are configured to drive the focus of the of the model to predetermined aspects of the conditional information embeddings to generate the conditional information embedding 46.

The noisy embedding 54 is concatenated with the conditional information embedding 46 to produce noisy concatenated input 56. The noisy concatenated input 56 is input to the denoising neural network 58 to generate the predicted sequence embedding 60. The predicted sequence embedding 60 is subsequently input to the sequence decoding neural network 62 to generate the predicted protein sequence 64 based upon the noisy concatenated input 56.

It will be appreciated that the denoising neural network 58 includes a temperature hyperparameter, which can be raised to increase the sensitivity to low probability candidates in the distribution and lowered to decrease this sensitivity. To optimize the temperature and improve the performance of the denoising neural network 58, some implementations may include a predicted Local Distance Difference Test (pLDDT) as a reward function that is used to vary the temperature hyperparameter of the denoising neural network 58. The pLDDT is a per residue confidence score, with values ranging between 0 and 100, that signifies the confidence of the prediction for each amino acid residue in the predicted protein sequence 64 relative to the Ca atoms, with values greater than 90 indicating high confidence, and values below 50 indicating low confidence. Other suitable reward functions may alternatively be adopted.

In addition to temperature parameter optimization, other implementations may include fine-tuning the weights of the DDPM to build model variants optimized for different downstream applications. This fine tuning is performed because a single set of weights will not be optimal for all downstream tasks. For instance, during initial training, the model learns to denoise sequence embeddings to reconstruct the complete original input sequence. Another, related application of the DDPM, however, would be to fix some amino acids in the output (i.e., deterministically enforce that the model outputs specific amino acids at specific output positions) and then fill in (in other words “inpaint”) the sequence between these deterministic positions. Such fixed amino acids can be selected for one or more reasons and via distinct, separate modeling and analyses or as part of the overall operation of the methodology. Though this application is similar to how the DDPM is trained, it is not identical, so the model will not be optimized for it. A variant of the DDPM that is further trained (“fine-tuned”) on this new task to update the weights learned during the initial training phase can thus be created to optimize its performance at the new inpainting task.

It will be appreciated that the potential fine tuning tasks available are not limited to the inpainting discussed in the previous paragraph. Other examples might include learning weights when certain conditional information is always provided as opposed to optionally provided as in the original training scheme, learning weights when certain conditional information is never provided as opposed to optionally provided as in the original training scheme, optimizing the model for generating sequences from specific families of proteins rather than the full known sequence and structural space that the model is initially trained on, fine-tuning the model with new, previously unused conditional information, and many others not listed here.

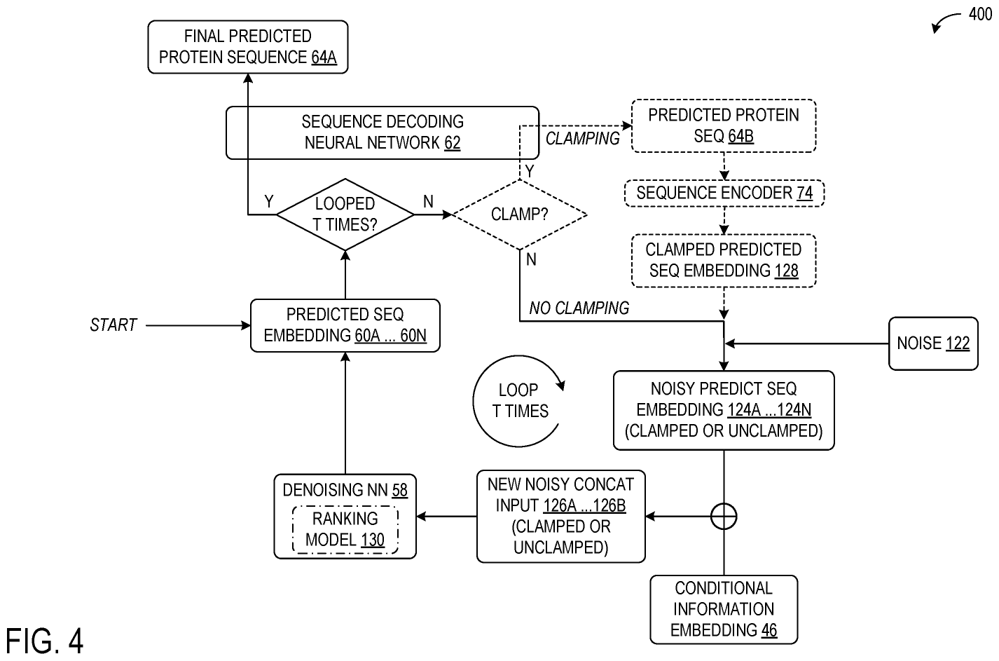

In some implementations, the processing circuitry 18 may send the predicted sequence embedding 60 through a denoising loop in which the predicted sequence embedding 60 undergoes one or more cycles of noising and denoising to improve the predicted protein sequence. FIG. 4 shows an example denoising loop 400 that is used to guide the DDPM 26 to an improved final predicted protein sequence that better resembles known protein sequences. Prior to decoding the predicted sequence embedding 60 via the sequence decoding neural network 62, it is determined whether the predicted sequence embedding 60 has traversed the denoising loop a predetermined number of (i.e., T) times. If not, it may then be determined whether the predicted sequence embedding 60 should be clamped, as described in detail below.

Continuing through the denoising loop, noise 122 is added to a first predicted sequence embedding 60A according to Equation 1, using the predicted sequence embedding 60A as x₀ and t=T−1 to generate a noisy predicted sequence embedding 124A. The noisy predicted sequence embedding 124A is concatenated with the conditional information embedding 46 to generate a new noisy concatenated input 126A that is input to the denoising neural network 58. The resultant new predicted sequence embedding 60B is then noised again using Equation 1, this time with t=T−2. The noisy predicted sequence embedding 124B is concatenated with the conditional information embedding 46 to generate a new noisy concatenated input 126B. The new noisy concatenated input 126B is passed through the denoising neural network 58 to produce a new predicted sequence embedding 60C.

This process of adding noise to the predicted sequence embedding 60, concatenating the resulting noisy predicted sequence embedding 124 with the conditional information embedding 46, and denoising the new noisy concatenated input 126 to generate a new predicted sequence embedding is repeated T times, dropping t by 1 each iteration up to and including the iteration where t=1. The noisy predicted sequence embedding that is produced when t=1 is concatenated with the conditioning embedding and denoised to produce a final predicted sequence embedding 60N, which is then decoded to yield the final predicted protein sequence 64A.

In some implementations, the predicted sequence embedding 60A may be clamped to prevent the final predicted protein sequence 64A from diverging too far from the set of known sequences. As shown in the dashed line in FIG. 4, the predicted sequence embedding 60A is input to the sequence decoding neural network 62 to generate a clamped predicted protein sequence 64B. The clamped predicted protein sequence 64B is input to the sequence encoder 74 to generate the clamped predicted sequence embedding 128. As with the unclamped predicted sequence embedding 60, noise 122 is then added to the clamped predicted sequence embedding 128 to generate the noisy predicted sequence embedding 124.

During inference, the generation procedure can be further conditioned on quantitative, experimentally derived information not available during initial training of the denoising diffusion probabilistic model. This allows for optimization of specific functional capabilities of proteins by allowing users to iterate between generating a set of protein candidates, evaluating the suitability of those candidates through laboratory experimentation, then using the results of that laboratory experimentation to conditionally generate proteins enriched in the desired capability.

Beyond evaluating the overall suitability of those candidates through laboratory studies, specific experimental efforts, or likewise, efforts can be employed to identify and then include consideration of additional potentially relevant conditional information that had not been included in the initial training of the denoising diffusion probabilistic model. The identification of such information can be informed and guided by the results or partial results of the generation procedure.

Formal methods, known in the general literature as computing the expected value of information, can be harnessed for guiding experimentation or selection of additional conditioning information. These methods employ information-theoretic or decision-theoretic analyses that consider current uncertainties, represented as probability distributions, and estimates of revised distributions after considering the results of experimentation or consideration of new information sources. In the most general implementations, the expected informational benefits are balanced with the costs required to perform the experiments or access additional information.

Considerations can extend beyond guidance on the value of performing real-world experiments or on accessing specific additional relevant information. Similar analyses can be employed to guide the selective allocation of computational resources via inferences about the expected value of continuing to compute to refine one or more aspects of the larger computation or to focus computational resources on specific regions of a formative protein. Beyond guiding the partition of resources to one or more phases of the base-level analyses, per the methods disclosed herein, estimates of the value of computation via formal or heuristic procedures can also guide allocation of computational resources to external modules for processing with separate, distinct analyses that provide information back to the generation process.

We note that heuristic or formal methods aimed at identifying the value of additional information can be used to guide engagement with a human expert for input such as topology, constraints of preferred bases in a generated protein sequence, or where additional computation might be focused. Graphical visualizations of final or partial results of denoising can be coupled with interactive affordances allowing for local or global human input on the generation of candidate proteins. Methods can enable a mix of machine and human initiatives, where the system employs heuristics or more formal value of information analyses to automatically pause to ask for input or where human experts can lean in with input during the generation process.

Quantitative information used to optimize a specific protein function is not introduced to the generative procedure via the encoders described in FIG. 2. Instead, it is introduced by using a ranking model 130 (shown in FIG. 4 in dashed-dot line) that is trained on that quantitative data to influence the generative procedure at each step t during the denoising process. This quantitative conditional process proceeds as follows: A model is trained to rank the experimentally evaluated proteins from least desirable (i.e., the protein that shows the lowest level of the target function) to most desirable (i.e., the protein that shows the highest level of the target function). To train this model, experimentally evaluated proteins are encoded using the sequence encoder 74, then the resultant embeddings are mapped to a number that correlates to the level of function of those proteins. During the inference phase, the trained ranking model 130 is used to calculate the probability that the noisy sequence embedding at timestep t−1 corresponds to a protein with a higher level of function than does the noisy sequence embedding at timestep t. From this calculation, the gradient shown below in Equation 3 is calculated.

$${∇}_{{x}_{t - 1}} p ( {y}_{t - 1} > {y}_{t} \vert {x}_{t - 1} , {x}_{t} ) \qquad ( 3 )$$

Where xₜ is the noisy sequence embedding at timestep t, xₜ₋₁ is the noisy sequence embedding at timestep t−1, yₜ is the predicted functionality of the noisy sequence at timestep t, and yₜ₋₁ is the predicted functionality of the noisy sequence at timestep t−1. The calculated gradient is then added to the noisy sequence embedding at timestep t−1, which pushes the embedding toward regions with higher probability for improved functionality.

The strategy described herein enables the DDPM 26 to further guide the generation of predicted protein sequences using information that was not available during model training time, i.e., data that results from specific protein engineering experiments. For example, a user may perform laboratory-based biochemical assays and experiments with proteins generated from the DDPM 26, discover that the batch is not optimal, train the ranking model 130 to identify features that determine more optimal proteins, then use that ranking model 130 to guide diffusion toward improved proteins in a subsequent round of inference. Additionally, the ranking model 130 can be trained on such experimentally derived data to focus on a narrow region of the protein sequence space that exhibits the target functionality.

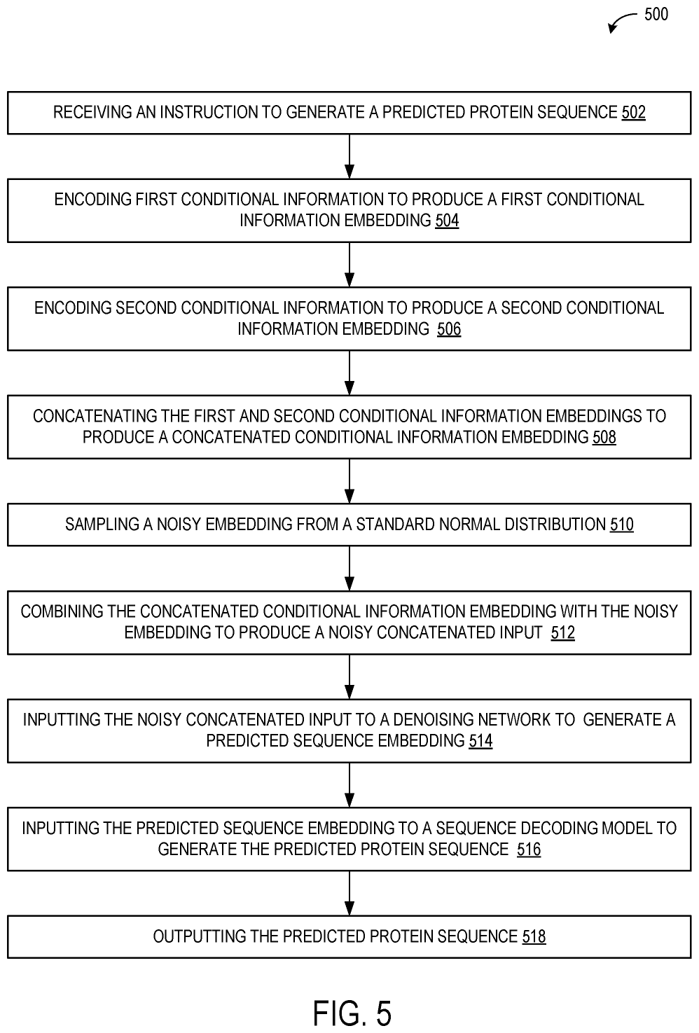

FIG. 5 shows a flowchart for a method 500 for conditionally generating protein sequences. Method 500 may be implemented by the hardware and software of computing system 10 described above, or by other suitable hardware and software. At step 502, the method 500 may include receiving an instruction to generate a predicted protein sequence having a target functionality. The instruction may include first conditional information and second conditional information associated with the target functionality of the predicted protein sequence, for example.

Proceeding from step 502 to step 504, the method 500 may further include encoding the first conditional information using a first encoder to produce a first conditional information embedding. Advancing from step 504 to step 506, the method 500 may further include encoding the second conditional information using a second encoder to produce a second conditional information embedding. As described above, the conditional information may be selected from protein structural information, textual information, chemical reaction information, and metadata associated with the input protein sequence. It will be appreciated that the conditional information embeddings may be encoded during the training phase and/or during the inference phase, depending on whether relevant new conditional information was acquired after the training phase.

Proceeding from step 506 to step 508, the method 500 may further include concatenating the first conditional information embedding and the second conditional information embedding to produce a concatenated conditional information embedding.

Advancing from step 508 to step 510, the method 500 may further include sampling a noisy embedding from a standard normal distribution. Continuing from step 510 to step 512, the method 500 may further include combining the concatenated conditional information embedding with the sampled noisy embedding to produce a noisy concatenated input.

Proceeding from step 512 to step 514, the method 500 may further include inputting the noisy concatenated input to a denoising neural network to cause the denoising neural network to generate a predicted sequence embedding. Continuing from step 514 to step 516, the method 500 may further include inputting the predicted sequence embedding to a sequence decoding neural network to generate the predicted protein sequence based upon the inputted predicted sequence embedding. Advancing from step 516 to step 518, the method 500 may include outputting the predicted protein sequence. The predicted protein sequence may be displayed in a user interface on a display.

In some embodiments, the method 500 may include iterating through a cycle of noising and denoising the predicted sequence embedding to guide the DDPM 26 to an improved final predicted protein sequence that better resembles known protein sequences. To prevent the final predicted protein sequence from diverging too far from the set of known protein sequences, the method 500 may include generating a clamped sequence embedding by passing the predicted protein sequence through the sequence encoder. The clamped sequence embedding may be noised according to the current timestep t and Equation 1. The method 500 may further include concatenating the noisy clamped sequence embedding with the concatenated conditional information embedding to generate a first noisy clamped concatenated input. The first noisy clamped concatenated input is then denoised to produce a clamped predicted sequence embedding. The method 500 may include repeating the process of adding noise to the newest clamped predicted sequence embedding, concatenating the resulting noisy clamped sequence embedding with the conditioning embedding, and denoising the noisy clamped concatenated input to generate a new clamped predicted sequence embedding T times, dropping t by 1 each iteration up to and including the iteration where t=1. The noisy clamped sequence embedding that is produced when t=1 is concatenated with the conditioning embedding and denoised to produce a final new predicted sequence embedding which is then decoded to yield the final predicted protein sequence

In some embodiments, the noised embedding from a current timestep and a noised embedding from the previous timestep are passed through a ranking module to predict the probability that the noised embedding from the current timestep exhibits a higher level of the target functionality than the noised embedding from the previous timestep. The gradient of this prediction is calculated according to Equation 3 and is used to update the noised embedding from the current timestep such that it is pushed toward representations that encode proteins with higher function.

The method 500 may further include a training phase. In the training phase, the method may include inputting a training protein sequence to a sequence encoder to convert the training protein sequence to a training sequence embedding that is a L×D matrix of numbers (where “L” is the length of the protein and “D” is a chosen dimensionality), choosing a random integer between 1 and T, adding noise sampled from the standard normal distribution to the training sequence embedding according to Equation 1 to produce a noisy training sequence embedding, encoding training conditional information from two or more conditional information classes via respective encoders to generate two or more training conditional information embeddings, inputting the two or more training conditional information embeddings to a feed forward neural network to generate a training conditional information embedding, combining the training conditional information embedding output from the feed forward neural network with the noisy training sequence embedding to produce a noisy training input, inputting the noisy training input to the denoising neural network to generate a predicted training sequence embedding, and inputting the predicted training sequence embedding to the sequence decoding neural network to generate a predicted training protein sequence based upon the inputted predicted training sequence embedding.

The denoising diffusion probabilistic model described herein provides a unified representation of multiple different classes of conditional information that can be used to aid in the conditional generation of a protein having a target functionality. This approach addresses challenges in fields of both computational protein optimization and computational protein design, as the predicted protein sequences may give rise to enhanced versions of existing proteins or represent de novo proteins having specific activities. The claimed latent diffusion model accepts all types of conditioning input as long as it is converted to a matrix or vector such that the shape of the input matches the latent diffusion space or can be represented using a ranking model that takes examples of the latent space as input. This feature enables the addition of conditional information to a trained model, as well as the ability to concatenate multiple types of conditional information from multiple sources and in various forms. As such, the claimed diffusion model has the potential to accelerate the development of computational protein engineering and make significant contributions to the study of molecular systems, drug design, drug discovery, and beyond.

In some embodiments, the methods and processes described herein may be tied to a computing system of one or more computing devices. In particular, such methods and processes may be implemented as a computer-application program or service, an application-programming interface (API), a library, and/or other computer-program products.

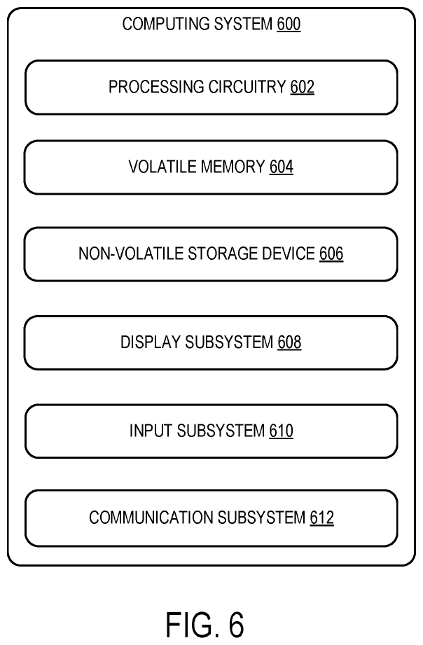

FIG. 6 schematically shows a non-limiting embodiment of a computing system 600 that can enact one or more of the methods and processes described above. Computing system 600 is shown in simplified form. Computing system 600 may embody the computing devices 14 and/or 16 described above and illustrated in FIG. 1. Computing system 600 may be included in one or more personal computers, server computers, tablet computers, home-entertainment computers, network computing devices, video game devices, mobile computing devices, mobile communication devices (e.g., smart phone), and/or other computing devices, and wearable computing devices such as smart wristwatches and head mounted augmented reality devices.

Computing system 600 includes processing circuitry 602, volatile memory 604, and a non-volatile storage device 606. Computing system 600 may optionally include a display subsystem 608, input subsystem 610, communication subsystem 612, and/or other components not shown in FIG. 1.

Processing circuitry 602 typically includes one or more logic processors, which are physical devices configured to execute instructions. For example, the logic processors may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic processor may include one or more physical processors configured to execute software instructions. Additionally or alternatively, the logic processor may include one or more hardware logic circuits or firmware devices configured to execute hardware-implemented logic or firmware instructions. Processors of the processing circuitry 602 may be single-core or multi-core, and the instructions executed thereon may be configured for sequential, parallel, and/or distributed processing. Individual components of the processing circuitry optionally may be distributed among two or more separate devices, which may be remotely located and/or configured for coordinated processing. For example, aspects of the computing system disclosed herein may be virtualized and executed by remotely accessible, networked computing devices configured in a cloud-computing configuration. In such a case, these virtualized aspects are run on different physical logic processors of various different machines, it will be understood. These different physical logic processors of the different machines will be understood to be collectively encompassed by processing circuitry 602.

Non-volatile storage device 606 includes one or more physical devices configured to hold instructions executable by the processing circuitry to implement the methods and processes described herein. When such methods and processes are implemented, the state of non-volatile storage device 606 may be transformed—e.g., to hold different data.

Non-volatile storage device 606 may include physical devices that are removable and/or built-in. Non-volatile storage device 606 may include optical memory, semiconductor memory, and/or magnetic memory, or other mass storage device technology. Non-volatile storage device 606 may include nonvolatile, dynamic, static, read/write, read-only, sequential-access, location-addressable, file-addressable, and/or content-addressable devices. It will be appreciated that non-volatile storage device 606 is configured to hold instructions even when power is cut to the non-volatile storage device 606.

Volatile memory 604 may include physical devices that include random access memory. Volatile memory 604 is typically utilized by processing circuitry 602 to temporarily store information during processing of software instructions. It will be appreciated that volatile memory 604 typically does not continue to store instructions when power is cut to the volatile memory 604.

Aspects of processing circuitry 602, volatile memory 604, and non-volatile storage device 606 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of computing system 600 typically implemented in software by a processor to perform a particular function using portions of volatile memory, which function involves transformative processing that specially configures the processor to perform the function. Thus, a module, program, or engine may be instantiated via processing circuitry 602 executing instructions held by non-volatile storage device 606, using portions of volatile memory 604. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

When included, display subsystem 608 may be used to present a visual representation of data held by non-volatile storage device 606. The visual representation may take the form of a graphical user interface (GUI). As the herein described methods and processes change the data held by the non-volatile storage device, and thus transform the state of the non-volatile storage device, the state of display subsystem 608 may likewise be transformed to visually represent changes in the underlying data. Display subsystem 608 may include one or more display devices utilizing virtually any type of technology. Such display devices may be combined with processing circuitry 602, volatile memory 604, and/or non-volatile storage device 606 in a shared enclosure, or such display devices may be peripheral display devices.

When included, input subsystem 610 may comprise or interface with one or more user-input devices such as a keyboard, mouse, touch screen, camera, or microphone.

When included, communication subsystem 612 may be configured to communicatively couple various computing devices described herein with each other, and with other devices. Communication subsystem 612 may include wired and/or wireless communication devices compatible with one or more different communication protocols. As non-limiting examples, the communication subsystem may be configured for communication via a wired or wireless local- or wide-area network, broadband cellular network, etc. In some embodiments, the communication subsystem may allow computing system 600 to send and/or receive messages to and/or from other devices via a network such as the Internet.

The following paragraphs provide additional description of aspects of the present disclosure. One aspect provides a computing system for conditional generation of protein sequences. The computing system may comprise processing circuitry that executes instructions using portions of associated memory to implement a denoising diffusion probabilistic model. The processing circuitry may be configured to receive an instruction to generate a predicted protein sequence having a target functionality, the instruction including first conditional information and second conditional information associated with the target functionality of the predicted protein sequence; concatenate a first conditional information embedding generated by a first encoder and a second conditional information embedding generated by a second encoder to produce a concatenated conditional information embedding, the first conditional information embedding representing the first conditional information and the second conditional information embedding representing the second conditional information; sample a noisy embedding from a distribution function; combine the concatenated conditional information embedding with the sampled noisy embedding to produce a noisy concatenated input; input the noisy concatenated input to a denoising neural network to cause the denoising neural network to generate a predicted sequence embedding; input the predicted sequence embedding to a decoding neural network to generate the predicted protein sequence based upon the inputted predicted sequence embedding; and output the predicted protein sequence.

In this aspect, additionally or alternatively, the distribution function may be a standard normal distribution.

In this aspect, additionally or alternatively, prior to inputting the predicted sequence embedding to the decoding neural network, the processing circuitry may send the predicted sequence embedding through a denoising loop in which noise is added to the predicted sequence embedding to generate a noisy predicted sequence embedding, the noisy predicted sequence embedding is concatenated with the conditional information embedding to generate a new noisy concatenated input, and the new noisy concatenated input is inputted to the denoising neural network to generate a new predicted sequence embedding.

In this aspect, additionally or alternatively, the denoising loop may be repeated a predetermined number of times to generate a final predicted sequence embedding, the final predicted sequence embedding may be input to a decoding neural network to generate a final predicted protein sequence, and the final predicted protein sequence may be output.

In this aspect, additionally or alternatively, prior to inputting the predicted sequence embedding to a decoding neural network to generate the predicted protein, the predicted protein sequence may be input to the sequence encoder to generate a clamped predicted sequence embedding, and the processing circuitry may send the clamped predicted sequence embedding through a denoising loop in which noise is added to the clamped predicted sequence embedding to generate a noisy clamped predicted sequence embedding, the noisy clamped predicted sequence embedding is concatenated with the conditional information embedding to generate a new noisy clamped concatenated input, and the new noisy clamped concatenated input is inputted to the denoising neural network to generate a new predicted sequence embedding.

In this aspect, additionally or alternatively, the denoising loop may be repeated a predetermined number of times to generate a final predicted sequence embedding, and the final predicted sequence embedding may be input to a decoding neural network to generate a final predicted protein sequence, and the final predicted protein sequence is output.

In this aspect, additionally or alternatively, the first conditional information may be selected from the group comprising: protein structural information, textual information, chemical reaction information, and metadata associated with the input protein sequence, and the second conditional information may be different from the first conditional information and may be selected from the group comprising: protein structural information, textual information, chemical reaction information, and metadata associated with the input protein sequence.

In this aspect, additionally or alternatively, in a training phase, the processing circuitry may be configured to receive a text instruction to generate a predicted protein sequence, the text instruction including a training protein sequence and training conditional information from two or more conditional information classes; input the training protein sequence to a sequence encoder to convert the training protein sequence to a training sequence embedding; add noise sampled from the distribution function to the training sequence embedding to produce a noisy training sequence embedding; encode the training conditional information via respective encoders to generate two or more training conditional information embeddings; input the two or more training conditional information embeddings to a feed forward neural network to generate a training conditional information embedding; combine the training conditional information embedding output from the feed forward neural network with the noisy training sequence embedding to produce a noisy training input; input the noisy training input to the denoising neural network to generate a predicted training sequence embedding; and input the predicted training sequence embedding to the sequence decoding neural network to generate a predicted training protein sequence based on the training protein sequence and the training conditional information.

In this aspect, additionally or alternatively, the denoising neural network may include a temperature hyperparameter and a predicted location distance different test to serve as a reward function to vary sensitivity of the temperature hyperparameter.

In this aspect, additionally or alternatively, a trained ranking model may calculate a probability that the noisy predicted sequence embedding corresponds to a protein sequence with a higher level of target functionality than that of another protein sequence embedding.

Another aspect provides a method for conditionally generating protein sequences. In an inference phase, the method may comprise receiving an instruction to generate a predicted protein sequence having a target functionality, the instruction including first conditional information and second conditional information associated with the target functionality of the predicted protein sequence; concatenating a first conditional information embedding generated by a first encoder and a second conditional information embedding generated by a second encoder to produce a concatenated conditional information embedding, the first conditional information embedding representing the first conditional information and the second conditional information embedding representing the second conditional information; sampling a noisy embedding from a distribution function; combining the concatenated conditional information embedding with the sampled noisy embedding to produce a noisy concatenated input; inputting the noisy concatenated input to a denoising neural network to cause the denoising neural network to generate a predicted sequence embedding; inputting the predicted sequence embedding to a sequence decoding neural network to generate the predicted protein sequence based on the inputted predicted sequence embedding; and outputting the predicted protein sequence.

In this aspect, additionally or alternatively, the distribution function may be a standard normal distribution.

In this aspect, additionally or alternatively, the method may further comprise, in a denoising loop prior to inputting the predicted sequence embedding to the decoding neural network, adding noise to the predicted sequence embedding to generate a noisy predicted sequence embedding, concatenating the noisy predicted sequence embedding with the conditional information embedding to generate a new noisy concatenated input, and inputting the new noisy concatenated input to the denoising neural network to generate a new predicted sequence embedding.

In this aspect, additionally or alternatively, the method may further comprise repeating the denoising loop a predetermined number of times to generate a final predicted sequence embedding, inputting the final predicted sequence embedding to a decoding neural network to generate a final predicted protein sequence, and outputting the final predicted protein sequence.

In this aspect, additionally or alternatively, the method may further comprise, prior to inputting the predicted sequence embedding to the decoding neural network, inputting the clamped predicted protein sequence to the sequence encoder to generate a clamped predicted sequence embedding, and sending the predicted sequence embedding through a denoising loop, the denoising loop including adding noise to the clamped predicted sequence embedding to generate a noisy clamped predicted sequence embedding, concatenating the noisy clamped predicted sequence embedding with the conditional information embedding to generate a new noisy clamped concatenated input, and inputting the new noisy clamped concatenated input to the denoising neural network to generate a new predicted sequence embedding.

In this aspect, additionally or alternatively, the method may further comprise repeating the denoising loop a predetermined number of times to generate a final predicted sequence embedding, inputting the final predicted sequence embedding to a decoding neural network to generate a final predicted protein sequence, and outputting the final predicted protein sequence.

In this aspect, additionally or alternatively, the method may further comprise selecting the first conditional information from the group comprising: protein structural information, textual information, chemical reaction information, and metadata associated with the input protein sequence; and selecting the second conditional information from the group comprising: protein structural information, textual information, chemical reaction information, and metadata associated with the target functionality of the input protein sequence, wherein the second conditional information is different from the first conditional information.

In this aspect, additionally or alternatively, the method may further comprise, in a training phase, inputting a training protein sequence to a sequence encoder to convert the training protein sequence from raw text data to a training sequence embedding, adding noise sampled from the standard normal distribution to the training sequence embedding to produce a noisy training sequence embedding, encoding training conditional information from two or more conditional information classes via respective encoders to generate two or more training conditional information embeddings, inputting the two or more training conditional information embeddings to a feed forward neural network to generate a training conditional information embedding, combining the training conditional information embedding output from the feed forward neural network with the noisy training sequence embedding to produce a noisy training input, inputting the noisy training input to the denoising neural network to generate a predicted training sequence embedding, and inputting the predicted training sequence embedding to the sequence decoding neural network to generate a predicted training protein sequence based upon the training protein sequence and the training conditional information.

In this aspect, additionally or alternatively, the method may further comprise including in the denoising neural network a temperature hyperparameter and a predicted local distance difference test to serve as a reward function to vary sensitivity of the temperature hyperparameter.

Another aspect provides a computing system for conditional generation of protein sequences. The computing system may comprise processing circuitry that executes instructions using portions of associated memory to implement a denoising diffusion probabilistic model. The processing circuitry may be configured to: receive an instruction to implement a de novo protein design approach to generate a predicted protein sequence having a target functionality, the instruction including first conditional information and second conditional information for the predicted protein sequence; encode the first conditional information for the target protein sequence using a first encoder to produce a first conditional information embedding; encode the second conditional information for the input protein sequence using a second encoder to produce a second conditional information embedding; concatenate the first conditional information embedding and the second conditional information embedding to produce a conditional information embedding; sample a noisy embedding from a standard normal distribution; combine the conditional information embedding with the sampled noisy embedding to produce a noisy concatenated input; input the noisy concatenated input to a denoising neural network to cause the denoising neural network to generate a predicted sequence embedding; input the predicted sequence embedding to a decoding neural network to generate the predicted protein sequence based upon the inputted predicted sequence embedding; input the predicted protein sequence to the sequence encoder to generate a clamped predicted sequence embedding; send the predicted sequence embedding through a denoising loop in which noise is added to the clamped predicted sequence embedding to generate a noisy clamped predicted sequence embedding, the noisy clamped predicted sequence embedding is concatenated with the conditional information embedding to generate a new noisy clamped concatenated input, and the new noisy clamped concatenated input is inputted to the denoising neural network to generate a new predicted sequence embedding; repeat the denoising loop a predetermined number of times to generate a final predicted sequence embedding; input the final predicted sequence embedding to the sequence decoding neural network to generate a final predicted protein sequence; and output the final predicted protein sequence.

“And/or” as used herein is defined as the inclusive or V, as specified by the following truth table:

| A | B | A ∨ B |
|---|---|---|
| True | True | True |
| True | False | True |
| False | True | True |
| False | False | False |

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computing system for conditional generation of protein sequences, the computing system comprising processing circuitry that executes instructions using portions of associated memory to implement a denoising diffusion probabilistic model, wherein, in an inference phase, the processing circuitry is configured to:
receive an instruction to generate a predicted protein sequence having a target functionality, the instruction including first conditional information and second conditional information associated with the target functionality of the predicted protein sequence;
concatenate a first conditional information embedding generated by a first encoder and a second conditional information embedding generated by a second encoder to produce a concatenated conditional information embedding, the first conditional information embedding representing the first conditional information and the second conditional information embedding representing the second conditional information;
sample a noisy embedding from a distribution function;
combine the concatenated conditional information embedding with the sampled noisy embedding to produce a noisy concatenated input;
input the noisy concatenated input to a denoising neural network to cause the denoising neural network to generate a predicted sequence embedding;
input the predicted sequence embedding to a decoding neural network to generate the predicted protein sequence based upon the inputted predicted sequence embedding; and
output the predicted protein sequence.

2. The computing system of claim 1, wherein
the distribution function is a standard normal distribution.

3. The computing system of claim 1, wherein,
prior to inputting the predicted sequence embedding to the decoding neural network, the processing circuitry sends the predicted sequence embedding through a denoising loop in which:
noise is added to the predicted sequence embedding to generate a noisy predicted sequence embedding,
the noisy predicted sequence embedding is concatenated with the conditional information embedding to generate a new noisy concatenated input, and
the new noisy concatenated input is inputted to the denoising neural network to generate a new predicted sequence embedding.

4. The computing system of claim 3, wherein
the denoising loop is repeated a predetermined number of times to generate a final predicted sequence embedding,
the final predicted sequence embedding is input to a decoding neural network to generate a final predicted protein sequence, and
the final predicted protein sequence is output.

5. The computing system of claim 1, wherein
prior to inputting the predicted sequence embedding to a decoding neural network to generate the predicted protein, the predicted protein sequence is input to the sequence encoder to generate a clamped predicted sequence embedding, and
the processing circuitry sends the clamped predicted sequence embedding through a denoising loop in which:
noise is added to the clamped predicted sequence embedding to generate a noisy clamped predicted sequence embedding,
the noisy clamped predicted sequence embedding is concatenated with the conditional information embedding to generate a new noisy clamped concatenated input, and
the new noisy clamped concatenated input is inputted to the denoising neural network to generate a new predicted sequence embedding.

6. The computing system of claim 5, wherein
the denoising loop is repeated a predetermined number of times to generate a final predicted sequence embedding, and
the final predicted sequence embedding is input to a decoding neural network to generate a final predicted protein sequence, and the final predicted protein sequence is output.

7. The computing system of claim 1, wherein
the first conditional information is selected from the group comprising: protein structural information, textual information, chemical reaction information, and metadata associated with the input protein sequence, and
the second conditional information is different from the first conditional information and is selected from the group comprising: protein structural information, textual information, chemical reaction information, and metadata associated with the input protein sequence.

8. The computing system of claim 1, wherein
in a training phase, the processing circuitry is configured to:
receive a text instruction to generate a predicted protein sequence, the text instruction including a training protein sequence and training conditional information from two or more conditional information classes;
input the training protein sequence to a sequence encoder to convert the training protein sequence to a training sequence embedding;
add noise sampled from the distribution function to the training sequence embedding to produce a noisy training sequence embedding;
encode the training conditional information via respective encoders to generate two or more training conditional information embeddings;
input the two or more training conditional information embeddings to a feed forward neural network to generate a training conditional information embedding;
combine the training conditional information embedding output from the feed forward neural network with the noisy training sequence embedding to produce a noisy training input;
input the noisy training input to the denoising neural network to generate a predicted training sequence embedding; and
input the predicted training sequence embedding to the sequence decoding neural network to generate a predicted training protein sequence based on the training protein sequence and the training conditional information.

9. The computing system of claim 1, wherein
the denoising neural network includes a temperature hyperparameter and a predicted location distance different test to serve as a reward function to vary sensitivity of the temperature hyperparameter.

10. The computing system of claim 3, wherein
a trained ranking model calculates a probability that the noisy predicted sequence embedding corresponds to a protein sequence with a higher level of target functionality than that of another protein sequence embedding.

11. A method for conditionally generating protein sequences, the method comprising, in an inference phase:
receiving an instruction to generate a predicted protein sequence having a target functionality, the instruction including first conditional information and second conditional information associated with the target functionality of the predicted protein sequence;
concatenating a first conditional information embedding generated by a first encoder and a second conditional information embedding generated by a second encoder to produce a concatenated conditional information embedding, the first conditional information embedding representing the first conditional information and the second conditional information embedding representing the second conditional information;
sampling a noisy embedding from a distribution function;
combining the concatenated conditional information embedding with the sampled noisy embedding to produce a noisy concatenated input;
inputting the noisy concatenated input to a denoising neural network to cause the denoising neural network to generate a predicted sequence embedding;
inputting the predicted sequence embedding to a sequence decoding neural network to generate the predicted protein sequence based on the inputted predicted sequence embedding; and
outputting the predicted protein sequence.

12. The method according to claim 11, wherein
the distribution function is a standard normal distribution.

13. The method according to claim 11, the method further comprising:
in a denoising loop prior to inputting the predicted sequence embedding to the decoding neural network:
adding noise to the predicted sequence embedding to generate a noisy predicted sequence embedding;
concatenating the noisy predicted sequence embedding with the conditional information embedding to generate a new noisy concatenated input; and
inputting the new noisy concatenated input to the denoising neural network to generate a new predicted sequence embedding.

14. The method according to claim 13, the method further comprising:
repeating the denoising loop a predetermined number of times to generate a final predicted sequence embedding;
inputting the final predicted sequence embedding to a decoding neural network to generate a final predicted protein sequence; and
outputting the final predicted protein sequence.

15. The method according to claim 11, the method further comprising:
prior to inputting the predicted sequence embedding to the decoding neural network, inputting the clamped predicted protein sequence to the sequence encoder to generate a clamped predicted sequence embedding; and
sending the predicted sequence embedding through a denoising loop, the denoising loop including:
adding noise to the clamped predicted sequence embedding to generate a noisy clamped predicted sequence embedding,
concatenating the noisy clamped predicted sequence embedding with the conditional information embedding to generate a new noisy clamped concatenated input, and
inputting the new noisy clamped concatenated input to the denoising neural network to generate a new predicted sequence embedding.

16. The method according to claim 15, the method further comprising:
repeating the denoising loop a predetermined number of times to generate a final predicted sequence embedding;
inputting the final predicted sequence embedding to a decoding neural network to generate a final predicted protein sequence; and
outputting the final predicted protein sequence.

17. The method according to claim 11, the method further comprising:
selecting the first conditional information from the group comprising: protein structural information, textual information, chemical reaction information, and metadata associated with the input protein sequence; and
selecting the second conditional information from the group comprising: protein structural information, textual information, chemical reaction information, and metadata associated with the target functionality of the input protein sequence, wherein
the second conditional information is different from the first conditional information.

18. The method according to claim 11, the method further comprising, in a training phase:
inputting a training protein sequence to a sequence encoder to convert the training protein sequence from raw text data to a training sequence embedding;
adding noise sampled from the standard normal distribution to the training sequence embedding to produce a noisy training sequence embedding;
encoding training conditional information from two or more conditional information classes via respective encoders to generate two or more training conditional information embeddings;
inputting the two or more training conditional information embeddings to a feed forward neural network to generate a training conditional information embedding;
combining the training conditional information embedding output from the feed forward neural network with the noisy training sequence embedding to produce a noisy training input;
inputting the noisy training input to the denoising neural network to generate a predicted training sequence embedding; and
inputting the predicted training sequence embedding to the sequence decoding neural network to generate a predicted training protein sequence based upon the training protein sequence and the training conditional information.

19. The method according to claim 11, the method further comprising:
including in the denoising neural network a temperature hyperparameter and a predicted local distance difference test to serve as a reward function to vary sensitivity of the temperature hyperparameter.

20. A computing system for conditional generation of protein sequences, the computing system comprising processing circuitry that executes instructions using portions of associated memory to implement a denoising diffusion probabilistic model, wherein, in an inference phase, the processing circuitry is configured to:
receive an instruction to implement a de novo protein design approach to generate a predicted protein sequence having a target functionality, the instruction including first conditional information and second conditional information for the predicted protein sequence;
encode the first conditional information for the target protein sequence using a first encoder to produce a first conditional information embedding;
encode the second conditional information for the input protein sequence using a second encoder to produce a second conditional information embedding;
concatenate the first conditional information embedding and the second conditional information embedding to produce a conditional information embedding;
sample a noisy embedding from a standard normal distribution;
combine the conditional information embedding with the sampled noisy embedding to produce a noisy concatenated input;
input the noisy concatenated input to a denoising neural network to cause the denoising neural network to generate a predicted sequence embedding;
input the predicted sequence embedding to a decoding neural network to generate the predicted protein sequence based upon the inputted predicted sequence embedding;
input the predicted protein sequence to the sequence encoder to generate a clamped predicted sequence embedding;
send the predicted sequence embedding through a denoising loop in which:
noise is added to the clamped predicted sequence embedding to generate a noisy clamped predicted sequence embedding,
the noisy clamped predicted sequence embedding is concatenated with the conditional information embedding to generate a new noisy clamped concatenated input, and
the new noisy clamped concatenated input is inputted to the denoising neural network to generate a new predicted sequence embedding;
repeat the denoising loop a predetermined number of times to generate a final predicted sequence embedding;
input the final predicted sequence embedding to the sequence decoding neural network to generate a final predicted protein sequence; and
output the final predicted protein sequence.
